(19) United States
(12) Patent Application Publication
(10) Pub. No.: US 2024/0423492 A1
(43) Pub. Date: Dec. 26, 2024

(54) DETECTION DEVICE AND TIMEPIECE

(71) Applicant: CASIO COMPUTER CO., LTD., Tokyo (JP)

(72) Inventor: Takashi SUENAGA, Tokyo (JP)

(73) Assignee: CASIO COMPUTER CO., LTD., Tokyo (JP)

(21) Appl. No.: 18/739,959

(22) Filed: Jun. 11, 2024

(30) Foreign Application Priority Data
Jun. 22, 2023 (JP) 2023-102477

(51) Int. Cl. A61B 5/024; G04B 47/06

(52) U.S. Cl. CPC A61B 5/02438; G04B 47/063

(57) ABSTRACT

A detection device including a main body case where at least part of a main surface has an opening, a board which is provided in the main body case, a first detection portion which is provided under the board in the main body case and detects biological information of a user, a window portion which is provided under the first detection portion and is in the opening of the main body case, and a second detection portion which is provided between the board and the window portion and detects, through the window portion, whether or not the detection device is in contact with or close to the skin of the user.

### CROSS-REFERENCE TO RELATED APPLICATION

This application is based upon and claims the benefit of priority from the prior Japanese Patent Application No. 2023-102477, filed Jun. 22, 2023, the entire contents of which are incorporated herein by reference.

### BACKGROUND

### 1. Technical Field

The present disclosure relates to a detection device that is used in a body wearable device such as a wristwatch which is used by being worn on an arm or the like, and a timepiece equipped with the detection device.

### 2. Description of the Related Art

For example, a wristwatch-type body wearable device that is used by being worn on an arm is known which detects a pulse only when it is worn on an arm so that unnecessary power consumption can be prevented, as described in Japanese Patent Application Laid-Open (Kokai) Publication No. 2016-129526.

The body wearable device described in Japanese Patent Application Laid-Open (Kokai) Publication No. 2016-129526 includes a pulse sensor which detects the pulse of a person wearing the body wearable device, and a body wearing sensor which detects whether or not the body wearable device has been worn on an arm. This body wearing sensor, which includes a plurality of metal pins provided projecting from the bottom of the body wearable device, judges whether or not the body wearable device has been worn on an arm by the plurality of metal pins coming in contact with the arm so as to detect electrostatic capacity changes occurring on the body.

### SUMMARY

An embodiment of the present disclosure is a detection device comprising: a main body case where at least part of a main surface has an opening; a board which is provided in the main body case; a first detection portion which is provided under the board in the main body case and detects biological information of a user; a window portion which is provided under the first detection portion and is in the opening of the main body case; and a second detection portion which is provided between the board and the window portion and detects, through the window portion, whether or not the detection device is in contact with or close to the skin of the user.

The above and further objects and novel features of the present invention will more fully appear from the following detailed description when the same is read in conjunction with the accompanying drawings. It is to be expressly understood, however, that the drawings are for the purpose of illustration only and are not intended as a definition of the limits of the invention.

### BRIEF DESCRIPTION OF THE DRAWINGS

FIG. 1 is a front view showing a first embodiment in which the present disclosure has been applied in a wristwatch;

FIG. 2 is a side view showing the three o'clock side of the wristwatch shown in FIG. 1;

FIG. 3 is a back view showing the bottom side of the wristwatch shown in FIG. 2;

FIG. 4 is an enlarged planar view showing the back cover of the wristwatch of FIG. 3 when viewed from the inner side of the wristwatch case;

FIG. 5 is an enlarged cross-sectional view showing a main portion of the back cover taken along the A-A arrow view in FIG. 4;

FIG. 6A is an enlarged top view of a sensor board shown in FIG. 4;

FIG. 6B is an enlarged bottom view of the sensor board shown in FIG. 4;

FIG. 7A is an enlarged planar view showing the back cover of FIG. 4 from which the sensor board has been detached and in which a spacing member has been arranged on the inner surface;

FIG. 7B is an enlarged planar view showing the back cover of FIG. 4 from which the sensor board and the spacing member have been detached;

FIG. 8 is an enlarged planar view showing a back cover when viewed from the inner side of a wristwatch case, in a second embodiment in which the present disclosure has been applied in a wristwatch;

FIG. 9 is an enlarged cross-sectional view showing a main portion of the back cover taken along the B-B arrow view in FIG. 8;

FIG. 10A is an enlarged top view of a sensor board shown in FIG. 8;

FIG. 10B is an enlarged bottom view of the sensor board shown in FIG. 8;

FIG. 11A is an enlarged planar view showing the back cover of FIG. 8 from which the sensor board has been detached and in which a spacing member has been arranged on the inner surface; and

FIG. 11B is an enlarged planar view showing the back cover of FIG. 8 from which the sensor board and the spacing member have been detached.

### DETAILED DESCRIPTION OF THE PREFERRED EMBODIMENTS

### First Embodiment

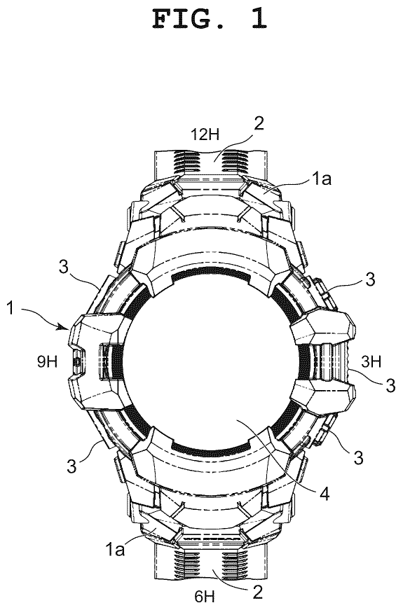

A first embodiment in which the present disclosure has been applied in a wristwatch will hereinafter be described with reference to FIG. 1 to FIG. 7.

This wristwatch includes a wristwatch case 1, as shown in FIG. 1 to FIG. 3. On the twelve o'clock side and six o'clock side of the wristwatch case 1, band attachment sections 1a are provided to which watch bands 2 are attached. In addition, on the two o'clock side, three o'clock side, four o'clock side, eight o'clock side, and ten o'clock side of the wristwatch case 1, switch sections 3 are provided. Here, for example, some of the switch sections 3 may be replaced by charging terminals.

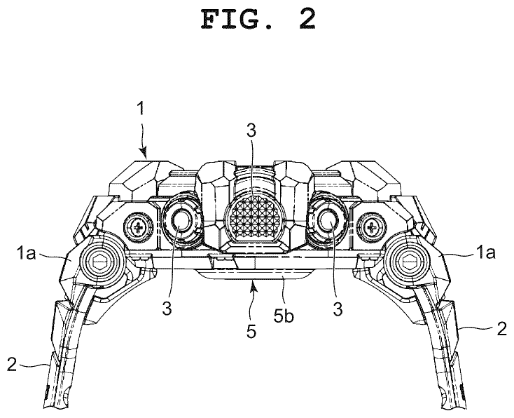

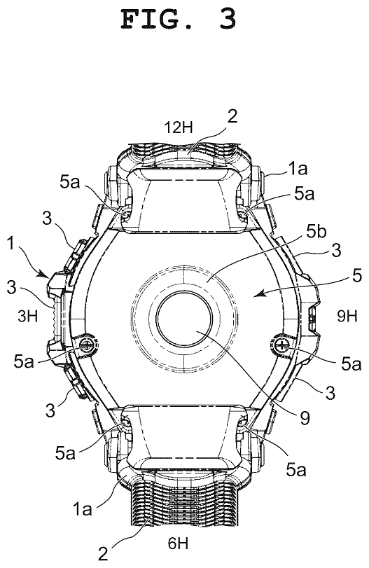

On the upper opening of the wristwatch case 1, a watch glass 4 (windshield) is provided via a glass packing (not shown), as shown in FIG. 1. In addition, to the lower side (back side) of the wristwatch case 1, a back cover 5 which is a lower windshield member is attached by a plurality of screws 5a via a waterproof ring (not shown), as shown in FIG. 2 and FIG. 3. This back cover 5 is formed of a highly rigid metal such as stainless steel or titanium alloy. Note that the back cover 5 is electrically connected to a GND (Ground) portion of a circuit board or the like, whereby its electric potential is maintained to be equal to GND and noise or the like is prevented from occurring.

Inside the wristwatch case 1, a timepiece module (not shown in the drawings) is provided. Although not shown in the drawings, this timepiece module includes various types of components required for timepiece functions, such as a timepiece movement which moves pointers to indicate the time, a flat-type display device which electro-optically displays information such as the time, date and day of the week, and the circuit board which controls and drives the timepiece movement and the display device.

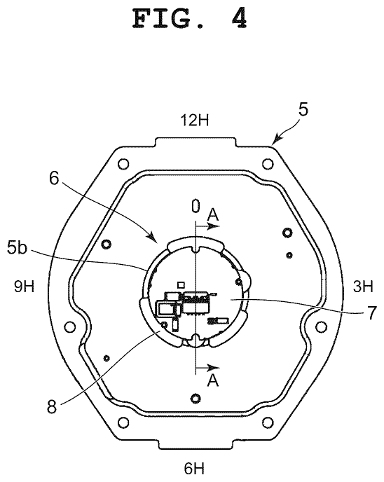

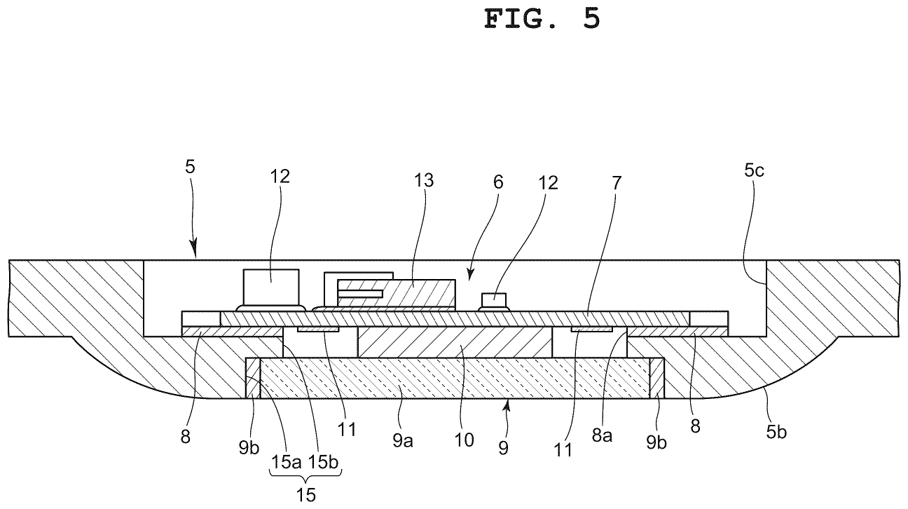

On the back cover 5 which is the lower windshield member for the wristwatch case 1, a detection device 6 is provided which detects biological information such as a pulse when the wristwatch case 1 is worn on an arm of a body, as shown in FIG. 4 and FIG. 5. This detection device 6 is arranged corresponding to a window section 9 of the back cover 5. In the present embodiment, on a substantially central portion of the back cover 5, a projection section 5b is formed which has a circular shape and project downward, as shown in FIG. 2 and FIG. 5.

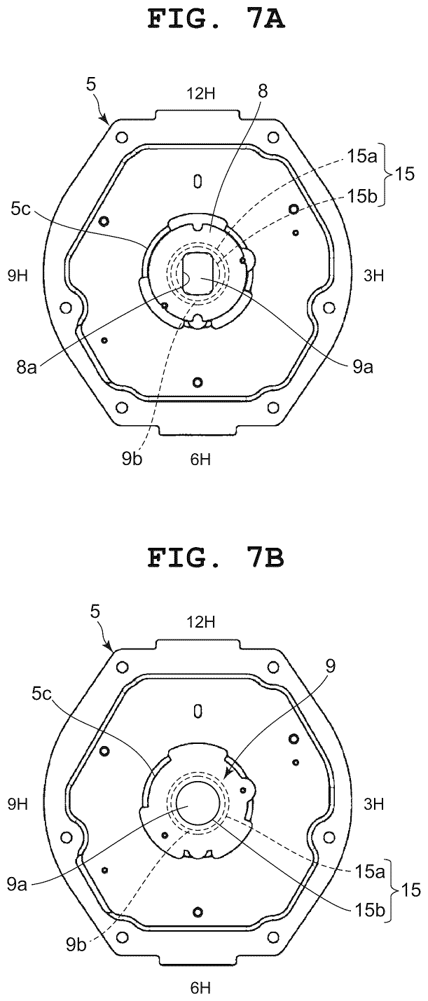

In the inner surface (upper surface) of the projection section 5b of the back cover 5, an attachment recess section 5c in which a sensor board 7 and a spacing member 8 are arranged is formed in a circular shape, as shown in FIG. 4 and FIG. 5. At the center of this attachment recess section 5c, the window section 9 is provided which has a circular shape and is light-transmissive, as shown in FIG. 5, FIG. 7A, and FIG. 7B. In the present embodiment, in the center of the attachment recess section 5c of the back cover 5, that is, in the center of the projection section 5b of the back cover 5, a through hole 15 is formed penetrating vertically. This through hole 15, which is an opening, includes a large-diameter hole 15a on the lower side and a small-diameter hole 15b on the upper side.

The window section 9 at the center of the projection section 5b of the back cover 5 has a structure where a window glass 9a has been fitted into the large-diameter hole 15a of the through hole 15 in the projection section 5b of the back cover 5 via a waterproof packing 9b, as shown in FIG. 5. As a result of this structure, in the window section 9, waterproofness and airtightness between the outer circumferential surface of the window glass 9a and the inner circumferential surface of the large-diameter hole 15a of the through hole 15 in the back cover 5 are ensured by the waterproof packing 9b, whereby high-pressure waterproofing is achieved. Note that the window glass 9a may be a transparent or translucent synthetic resin such as polymethyl methacrylate (PMMA), polycarbonate (PC), or ABS (Acrylonitrile Butadiene Styrene).

On the other hand, the detection device 6 includes a sensor board 7, as shown in FIG. 4 and FIG. 5. This sensor board 7, which has a disc-like shape, is attached to the inside of the attachment recess section 5c in the inner surface (upper surface) of the back cover 5 via the insulative spacing member 8 such as resin, as shown in, for example, FIG. 5 and FIG. 7A. As a result of this structure, by the spacing member 8, the sensor board 7 is attached to the inner surface of the attachment recess section 5c of the back cover 5 in a non-contact state where the sensor board 7 does not come in contact with the back cover 5. Note that the spacing member 8 should preferably have adhesion layers on its principal surfaces. As a result, the sensor board 7 can be fixed in the small space with the simple structure.

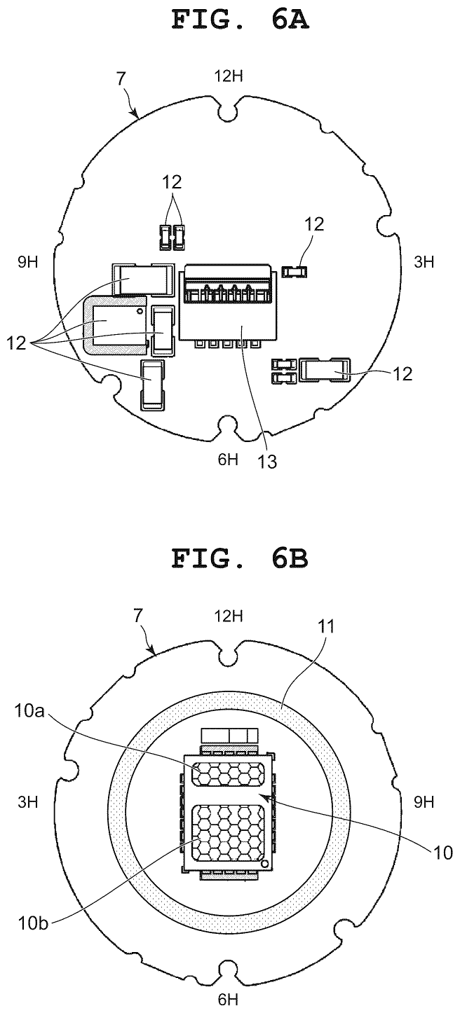

The undersurface of this sensor board 7 is provided with a pulse sensor 10 which is a first detection section for measuring a pulse that is biological information, and a body wearing sensor 11 which is a second detection section for judging whether or not the wristwatch case 1 has been worn on an arm, as shown in FIG. 6B. The pulse sensor 10 includes a light emitting element 10a and a light receiving element 10b provided on a substantially central portion of the undersurface of the sensor board 7. This pulse sensor 10 has a substantially rectangular plate shape which is long in the twelve o'clock direction and the six o'clock direction, and is arranged corresponding to a portion of the window glass 9a of the window section 9 corresponding to the small-diameter hole 15b of the through hole 15 formed in the projection section 5b of the back cover 5.

In the present embodiment, the light emitting element 10a of the pulse sensor 10 is an LED (Light-Emitting Diode) which emits light of a green wavelength (G) of about 550 nm, and is provided in an upper area corresponding to one-fourth of the pulse sensor 10 having a substantially rectangular shape, as shown in, for example, FIG. 6B. On the other hand, the light receiving element 10b is a photo diode or a photo transistor which receives reflected light resulting from light of the green wavelength emitted by the light emitting element 10a and applied to the skin of an arm, and is provided in a lower area corresponding to three-fourths of the pulse sensor 10 having a substantially rectangular shape.

The light emitting element 10a and light receiving element 10b of this pulse sensor 10 are arranged corresponding to a substantially central portion of the window glass 9a of the window section 9 corresponding to the small-diameter hole 15b of the through hole 15 formed in the projection section 5b of the back cover 5, as shown in FIG. 5 and FIG. 6B. As a result, the pulse sensor 10 is structured such that the light emitting element 10a emits light of the green wavelength, and this light of the green wavelength is applied to the skin of an arm via the window glass 9a of the window section 9.

Also, this pulse sensor 10 is structured such that part of light of the green wavelength applied to the skin of an arm is absorbed into capillary vessels under the skin of the arm, the remaining light not absorbed by the capillary vessels is reflected and received by the light receiving element 10b via the window glass 9a of the window section 9 of the back cover 5, and a pulse is measured on the basis of change in the received amount of reflected light, as shown in FIG. 5 and FIG. 6B.

On the other hand, the body wearing sensor 11 is an electrostatic capacity sensor that detects an electrostatic capacity change higher than a predetermined threshold value which occurs by the wristwatch case 1 coming in contact with or approaching a body, as shown in FIG. 5 and FIG. 6B. For example, this body wearing sensor 11 is copper foil provided on the undersurface of the sensor board 7 while surrounding the circumference of the pulse sensor 10 in a ring shape. Also, this body wearing sensor 11 is arranged corresponding to a portion of the window glass 9a of the window section 9 corresponding to the small-diameter hole 15b of the through hole 15 formed in the projection section 5b of the back cover 5. Note that the material of the body wearing sensor 11 is not limited to copper foil and may be various types of metallic materials and alloy materials.

That is, because of the structure where the window glass 9a of the window section 9 is fitted into the large-diameter hole 15a of the through hole 15 formed in the projection section 5b of the back cover 5 via the waterproof packing 9b, the outer circumference of the window glass 9a is formed longer than the outer circumference of the body wearing sensor 11, as shown in FIG. 5. As a result, when the wristwatch case 1 is worn on an arm and the back cover 5 which is a component mounting member is arranged on the arm, the body wearing sensor 11 detects an electrostatic capacity change higher than a predetermined threshold value, which occurs by the wristwatch case 1 coming in contact with or approaching a body, through the portion of the window glass 9a of the window section 9 corresponding to the small-diameter hole 15b of the through hole 15, and thereby detects that the wristwatch case 1 has been worn on the arm.

On the upper surface of the sensor board 7, a plurality of chip components 12 and a connector 13 are provided, as shown in FIG. 6A. These chip components 12 are electrically connected to the pulse sensor 10 via through holes (not shown in the drawings) in the sensor board 7 by wiring (not shown in the drawings) formed by pattern formation on the sensor board 7. As a result, these chip components 12 drive the light emitting element 10a of the pulse sensor 10, acquire change in the amount of light received by the light receiving element 10b as pulse information, and calculate a pulse by arithmetic processing based on the acquired pulse information.

In addition, these plural chip components 12 are electrically connected to the connector 13 on the upper surface of the sensor board 7 by wiring (not shown in the drawings) formed by pattern formation on the sensor board 7, as shown in FIG. 6A. As a result, the pulse sensor 10 is electrically connected to the connector 13 via the plurality of chip components 12. The body wearing sensor 11 is electrically connected to the connector 13 on the upper surface of the sensor board 7 via a through hole (not shown in the drawings) in the sensor board 7 by wiring (not shown in the drawings) formed by pattern formation on the sensor board 7, as shown in FIG. 6A and FIG. 6B.

This connector 13 is electrically connected to the circuit board (not shown in the drawings) for the timepiece module (not shown in the drawings) in the wristwatch case 1 by a flexible wiring board (not shown in the drawings). As a result, the timepiece module is structured such that, by the flat-type display device (not shown in the drawings) being electrically connected to the circuit board for the timepiece module, pulse information detected by the pulse sensor 10 is electro-optically displayed on the flat-type display device as a pulse rate.

In the present embodiment, the spacing member 8 is arranged between the sensor board 7 and the back cover 5, as shown in FIG. 5 and FIG. 7A. This spacing member 8 is, for example, made of an insulative synthetic resin and has a disc-like shape whose size is substantially equal to that of the sensor board 7. Also, this spacing member 8 is provided with a sensor insertion hole 8a in which the pulse sensor 10 and part of the body wearing sensor 11 are arranged opposing the window section 9 of the back cover 5. The sensor insertion hole 8a is formed penetrating the spacing member 8 vertically, and has a rectangular shape corresponding to the pulse sensor and part of the body wearing sensor 11.

In the present embodiment, when inserted into the sensor insertion hole 8a of the spacing member 8, the pulse sensor is arranged in the small-diameter hole 15b of the through hole 15 formed in the projection section 5b of the back cover with the undersurface of the pulse sensor 10 being in contact with or being positioned close to the inner surface (upper surface) of the window glass 9a of the window section 9, as shown in FIG. 5. Also, when the pulse sensor 10 is inserted into the sensor insertion hole 8a of the spacing member 8, the parts of the body wearing sensor 11 other than the above-described part of the body wearing sensor 11 inserted into the sensor insertion hole 8a are pressed against the upper surface of the spacing member 8.

As a result of this structure, the body wearing sensor 11 is arranged corresponding to the small-diameter hole 15b of the through hole 15 in the window section 9 of the back cover such that, by the insulative spacing member 8, this body wearing sensor 11 does not come in contact with the window glass 9a while being in a non-contact state with respect to the back cover 5, and the main surface of this body wearing sensor 11 is not opposed to the back cover 5 (that is, the electric field is not blocked by the back cover 5), as shown in FIG. 5. Accordingly, although the back cover 5 is made of a metal such as stainless steel or titanium alloy, the body wearing sensor 11 can detect an electrostatic capacity change higher than a predetermined threshold value which occurs by the wristwatch case 1 coming in contact with or approaching a body.

Next, the use of the wristwatch according to the first embodiment is described.

Normally, this wristwatch according to the first embodiment is used by the wristwatch case 1 being worn on an arm. In this state, information such the time, date and day of the week can be viewed by the timepiece module (not shown in the drawings) in the wristwatch case 1 through the watch glass 4. Here, when biological information such as a pulse is to be detected by the detection device 6, the wristwatch is first set to be in a detection mode by one of the plurality of switch sections 3 being selected and operated.

When detection by the detection device 6 is started, the body wearing sensor 11 of the detection device 6 detects an electrostatic capacity change higher than a predetermined threshold value, which occurs by the wristwatch case 1 coming in contact with or approaching a body, through the window section 9 of the back cover 5, and thereby judges whether or not the wristwatch case 1 has been worn on an arm. When a judgment that the wristwatch case 1 has not been worn on an arm is made by the body wearing sensor 11, pulse measurement by the pulse sensor 10 is not performed, so that unnecessary power consumption is prevented. Conversely, when a judgment that the wristwatch case 1 has been worn on an arm is made by the body wearing sensor 11, the pulse sensor 10 of the detection device 6 starts pulse measurement.

When pulse measurement by the pulse sensor 10 is started, the light emitting element 10a of the pulse sensor 10 emits light of the green wavelength, and this light of the green wavelength is applied to the skin of the arm through the window glass 9a of the window section 9 provided in the back cover 5. Part of this applied light of the green wavelength is absorbed by capillary vessels under the skin of the arm, and the remaining light which has not been absorbed into the capillary vessels is reflected.

This reflected light is received by the light receiving element 10b of the pulse sensor 10 through the window glass 9a of the window section 9 of the back cover 5. Then, on the basis of change in the amount of light received by this light receiving element 10b, the pulse sensor 10 and the control section (not shown in the drawings) on the circuit board for the timepiece module performs arithmetic processing, and thereby calculates a pulse. The pulse information calculated thereby is sent to the circuit board for timepiece module (not shown in the drawings) in the wristwatch case 1 through the connector 13 and the flexible wiring board (not shown in the drawings), and displayed on the display device (not shown in the drawings) of the timepiece module as a pulse rate.

As described above, the detection device 6 of the wristwatch according to the first embodiment includes the back cover 5 which is a main body case where at least part of the main surface has an opening, the sensor board 7 provided in the back cover 5, the pulse sensor 10 which is a first detection section that is provided under the sensor board 7 in the back cover 5 and detects biological information, the window section 9 which is provided under the pulse sensor 10 and is in the opening of the back cover 5, and the body wearing sensor 11 which is a second detection section that is provided between the sensor board 7 and the window section 9 and detects, through the window section 9, whether or not the detection device 6 is in contact with or close to the skin of the user. As a result of this structure, the detection sensitivity of the body wearing sensor 11 serving as the second detection section can be ensured.

For example, in the case of the body wearable device described in Japanese Patent Application Laid-Open (Kokai) Publication No. 2016-129526, there is a problem in that, since the plurality of metal pins of the body wearing sensor that comes in contact with an arm is provided projecting outward from the bottom of the body wearable device, these metal pins of the body wearing sensor corrode or are worn out due to overuse, which decreases the detection sensitivity for detecting electrostatic capacity changes that occur on a body. However, the detection device 6 of the wristwatch according to the first embodiment does not have this problem.

That is, in the case of the detection device 6 of this wristwatch, the body wearing sensor 11 is arranged without being exposed to the outside of the back cover 5. As a result, the corrosion and wear of the body wearing sensor 11 is prevented, and the detection sensitivity of the body wearing sensor 11 is ensured. In addition, the waterproof structure is not complicated, and waterproofing is ensured by this simple waterproof structure. Also, the body wearing sensor 11 detects whether or not the back cover 5 is in contact with a body through the window section 9 of the back cover 5. As a result of this structure, the body wearing sensor 11 can be arranged corresponding to the window section 9 of the back cover 5 while being in a non-contact state with respect to the back cover 5.

Accordingly, in the case of the detection device 6 of this wristwatch, although the back cover 5 is made of metal, the body wearing sensor 11 corresponding to the window section 9 can unfailingly and favorably detect whether or not the wristwatch case 1 has been worn on a body. As a result of this structure, pulse measurement by the pulse sensor 10 is performed only when the wristwatch case 1 is worn on a body, whereby unnecessary power consumption is prevented.

In the case of the detection device 6 of this wristwatch, since the window section 9 of the back cover 5 has the structure where the window glass 9a has been fitted into the through hole in the back cover 5 together with the waterproof packing 9b, waterproofness is ensured with the simple waterproof structure. More specifically, this window section 9 has the structure where the through hole 15 of the back cover 5 includes the large-diameter hole 15a on the lower side and the small-diameter hole 15b on the upper side, and the window glass 9a has been fitted into the large-diameter hole 15a on the lower side via the waterproof packing 9b. Accordingly, the waterproofness of the window section 9 is ensured by the waterproof packing 9b.

Also, in the case of the detection device 6 of this wristwatch, the body wearing sensor 11 is an electrostatic capacity sensor which detects an electrostatic capacity change occurring on a body. Accordingly, although the back cover 5 is made of metal, an electrostatic capacity change higher than a predetermined threshold value which occurs by the wristwatch case 1 coming in contact with or approaching a body is unfailingly detected through the window section 9 of the back cover 5 by the body wearing sensor 11 provided corresponding to the window glass 9a of the window section 9 of the back cover 5, or more specifically, the portion of the window glass 9a corresponding to the small-diameter hole 15b of the through hole 15. As a result of this structure, a judgment as to whether or not the back cover 5 of the wristwatch case 1 is in contact with a body can be accurately and favorably made.

Moreover, in the case of the detection device 6 of this wristwatch, the back cover 5 which is a component mounting member is made of a metal such as stainless steel or titanium alloy. Accordingly, the rigidity of the back cover 5 is ensured, and the strength of the entire wristwatch case 1 is increased. As a result, impact resistance and waterproofness can be ensured and, although the back cover 5 is made of metal, a judgment as to whether or not the back cover 5 of the wristwatch case 1 is in contact with a body can be accurately and favorably made by the body wearing sensor 11 provided corresponding the portion of the window glass 9a corresponding to the small-diameter hole 15b of the through hole 15 in the window section 9 of the back cover 5.

Furthermore, in the detection device 6 of this wristwatch, the body wearing sensor 11 is provided on the sensor board 7. Therefore, although the back cover 5 is made of metal, the body wearing sensor 11 can be arranged corresponding to the window section 9 of the back cover 5 while being in a non-contact state with respect to the back cover 5. As a result of this structure, by the body wearing sensor 11, an electrostatic capacity change higher than a predetermined threshold value, which occurs by the wristwatch case 1 coming in contact with or approaching a body, is favorably detected, whereby a judgment whether or not the back cover 5 of the wristwatch case 1 is in contact with the body is unfailingly and favorably made.

In the case of the detection device 6 of this wristwatch, the body wearing sensor 11 surrounds the circumference of the pulse sensor 10. As a result of this structure, an electrostatic capacity change higher than a predetermined threshold value, which occurs by the wristwatch case 1 coming in contact with or approaching a body, is unfailingly and favorably detected by the body wearing sensor 11 without body pulse measurement by the pulse sensor 10 being affected, whereby the pulse of the body is accurately detected by the pulse sensor 10, and whether or not the body is in contact with the back cover 5 is reliably detected through the window section 9 of the back cover 5.

Also, in the detection device 6 of this wristwatch, the insulative spacing member 8 is arranged between the back cover which is a component mounting member and the sensor board 7. In addition, this spacing member 8 is provided with the sensor insertion hole 8a into which the pulse sensor 10 is inserted in a manner to be positioned corresponding to the window section 9. As a result of this structure, by the spacing member 8, the body wearing sensor 11 is reliably prevented from coming in contact with the back cover 5 and favorably positioned corresponding to the portion of the window glass 9a of the window section 9 of the back cover 5 corresponding to the small-diameter hole 15b of the through hole 15, whereby an electrostatic capacity change which occurs on a body is accurately and favorably detected by the body wearing sensor 11.

In the case of the detection device 6 of this wristwatch, the pulse sensor 10 is inserted into the sensor insertion hole 8a of the spacing member 8. Accordingly, the light emitting element 10a and the light receiving element 10b of the pulse sensor 10 are arranged in the small-diameter hole 15b of the through hole 15 in the window section 9 of the back cover 5, and unfailingly and favorably positioned corresponding to the portion of the window glass 9a corresponding to the small-diameter hole 15b. As a result of this structure, pulse measurement is accurately and favorably performed by the light emitting element 10a and light receiving element 10b of the pulse sensor 10.

Also, in the detection device 6 of this wristwatch, the body wearing sensor 11 is electrically connected to the connector 13 on the upper surface of the sensor board 7 by the wiring (not shown in the drawings) formed by pattern formation on the sensor board 7, whereby the body wearing sensor 11 provided on the undersurface of the sensor board 7 and the connector 13 provided on the upper surface of the sensor board 7 are electrically connected to each other reliably and favorably.

In the case of the detection device 6 of this wristwatch, by the wiring (not shown in the drawings) formed by pattern formation on the sensor board 7, the pulse sensor 10 is connected to the plurality of chip components 12 provided on the upper surface of the sensor board 7, and the these chip components 12 are electrically connected to the connector 13. As a result of this structure, the pulse sensor 10 provided on the undersurface of the sensor board 7 and the connector 13 provided on the upper surface of the sensor board 7 are electrically connected to each other reliably and favorably.

Thus, in the detection device 6 of this wristwatch, the connector 13 of the sensor board 7 to which the body wearing sensor 11 and the pulse sensor 10 have been electrically connected is electrically and unfailingly connected to the circuit board (not shown in the drawings) for the timepiece module in the wristwatch case 1 by the flexible wiring board (not shown in the drawings). As a result of this structure, an instruction to perform pulse measurement is provided to the pulse sensor 10 by the body wearing sensor 11, and pulse information (pulse rate) measured by the pulse sensor 10 is favorably displayed on the display device (not shown in the drawings) of the timepiece module.

In the above-described first embodiment, the body wearing sensor 11 is provided on the undersurface of the sensor board 7. However, the present disclosure is not limited thereto, and the body wearing sensor 11 may be provided on the upper surface of the sensor board 7. In this case, the body wearing sensor 11 is provided on the upper surface of the sensor board 7 while surrounding an area corresponding to the pulse sensor and being positioned corresponding to the window section 9.

### Second Embodiment

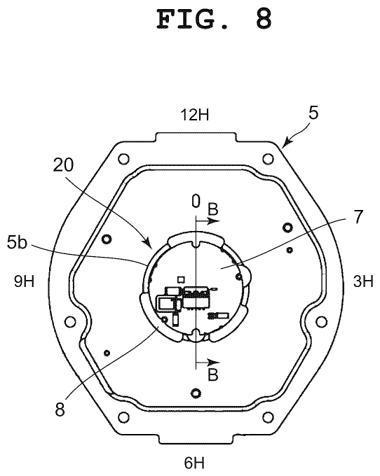

Next, a second embodiment in which the present disclosure has been applied in a wristwatch is described with reference to FIG. 8 to FIG. 11. Note that sections that are the same as those of the first embodiment shown in FIG. 1 to FIG. 7 are described using the same reference numerals.

This wristwatch has the same structure as the first embodiment except that a body wearing sensor 21 of a detection device 20 is provided on the window section 9 of the back cover 5.

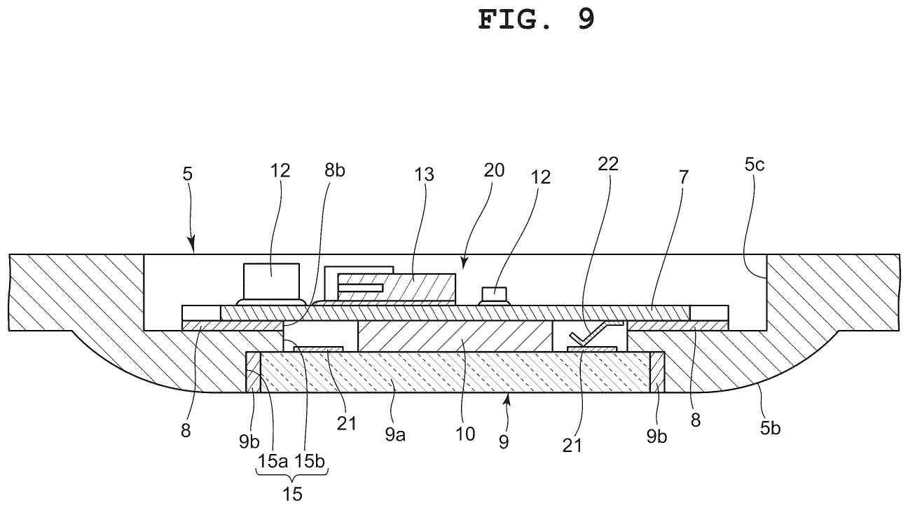

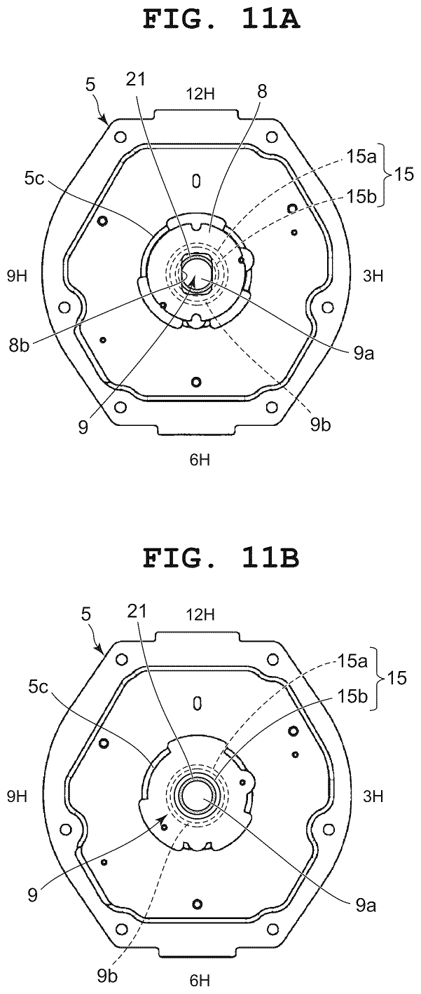

More specifically, the body wearing sensor 21 is an electrostatic capacity sensor for detecting an electrostatic capacity change that occurs on a body, as in the first embodiment. This body wearing sensor 21 is copper foil provided on the inner surface (upper surface) of a portion of the window glass 9a corresponding to the small-diameter hole 15b of the through hole 15 in the window section 9 while surrounding the circumference of the pulse sensor 10 in a ring shape, as shown in FIG. 9 and FIG. 11B. Note that the material of the body wearing sensor 21 is not limited to copper foil and may be various types of metallic materials and alloy materials.

In the present embodiment, the window glass 9a is made of insulative glass and has a disc-like shape whose size enables it to be fitted into the large-diameter hole 15a of the through hole 15 via the waterproof packing 9b, as shown in FIG. 9 and FIG. 11B. As a result, the body wearing sensor 21 is provided in a ring shape on the upper surface of the portion of the window glass 9a corresponding to the small-diameter hole 15b of the through hole 15 while having a size enabling the body wearing sensor 21 to surround the circumference of the pulse sensor 10 along the circumference of the pulse sensor 10.

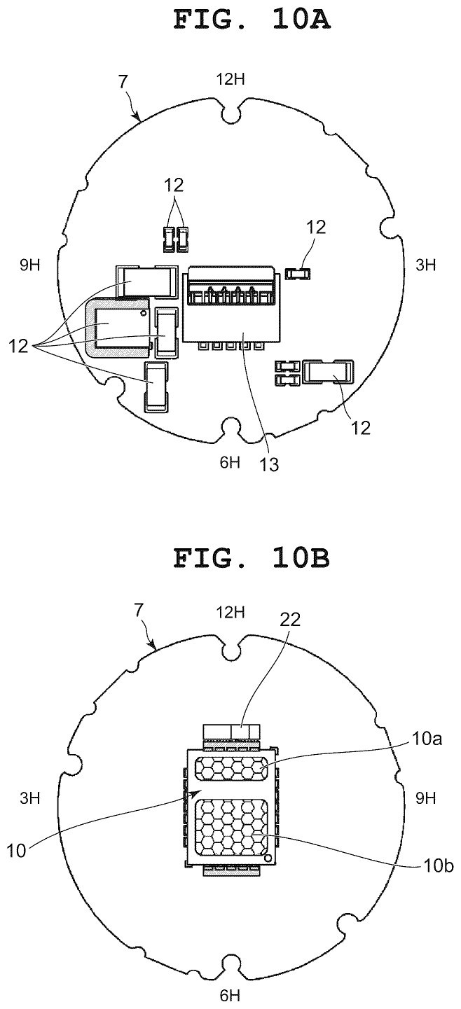

In the present embodiment, on the undersurface of the sensor board 7 provided with the pulse sensor 10, a connection member 22 is provided corresponding to the body wearing sensor 21 on the window section 9 of the back cover 5, as shown in FIG. 9 and FIG. 10B. This connection member 22 is a spring member such as a flat spring, and is arranged close to the upper side of the pulse sensor 10. As a result, when the sensor board 7 is arranged on the inner surface (upper surface) of the attachment recess section 5c of the back cover 5, the connection member 22 resiliently comes in contact with the body wearing sensor 21 and is electrically connected thereto. Also, this connection member 22 is electrically connected to the connector 13 on the upper surface of the sensor board 7 by wiring (not shown in the drawings) formed by pattern formation on the sensor board 7 via a through hole (not shown in the drawings) formed in the sensor board 7, as shown in FIG. 9, FIG. 10A, and FIG. 10B. The connector 13 is electrically connected to the circuit board (not shown in the drawings) for the timepiece module in the wristwatch case 1 by the flexible wiring board (not shown in the drawings), as in the first embodiment.

In the area between the sensor board 7 and the back cover 5, the spacing member 8 is arranged, as shown in FIG. 9 and FIG. 11A. This spacing member 8 is, for example, made of an insulative synthetic resin and has a disc-like shape whose size is substantially equal to that of the sensor board 7, as in the first embodiment. Also, this spacing member 8 is provided with a component insertion hole 8b into which the pulse sensor 10 and the connection member 22 are inserted opposing the window section 9 of the back cover 5. The component insertion hole 8b is formed penetrating the spacing member 8 vertically, and has a rectangular shape corresponding to the pulse sensor 10 and the body wearing sensor 22.

In the present embodiment, the body wearing sensor 21 is arranged such that, when the pulse sensor 10 and the connection member 22 are arranged in the component insertion hole 8b of the spacing member 8, the connection member 22 is resiliently pressed against and electrically connected to this body wearing sensor 21 while being in a non-contact state where the connection member 22 does not come in contact with the back cover 5, as shown in FIG. 9. As a result of this structure, although the back cover 5 is made of a metal such as stainless steel or titanium alloy, the body wearing sensor 11 can detect an electrostatic capacity change higher than a predetermined threshold value which occurs by the wristwatch case 1 coming in contact with or approaching a body, without being affected by the back cover 5.

Next, the use of the wristwatch according to the second embodiment is described.

Normally, this wristwatch according to the second embodiment is used by the wristwatch case 1 being worn on an arm, as in the case of the first embodiment. In this state, information such as the time, date and day of the week can be viewed by the timepiece module (not shown in the drawings) in the wristwatch case 1 through the watch glass 4. Here, when biological information such as a pulse is to be detected by the detection device 20, the wristwatch is first set to be in the detection mode by one of the plurality of switch sections 3 being selected and operated.

When detection by the detection device 20 is started, first, the body wearing sensor 21 of the detection device 20 detects an electrostatic capacity change higher than a predetermined threshold value, which occurs by the wristwatch case 1 coming in contact with or approaching a body, through the window section 9 of the back cover 5, and thereby judges whether or not the wristwatch case 1 has been worn on an arm. When a judgment that the wristwatch case 1 has not been worn on an arm is made by the body wearing sensor 21, pulse measurement by the pulse sensor 10 is not performed, so that unnecessary power consumption is prevented. Conversely, when a judgment that the wristwatch case 1 has been worn on an arm is made by the body wearing sensor 21, the pulse sensor 10 of the detection device 20 starts pulse measurement.

When pulse measurement by the pulse sensor 10 is started, the light emitting element 10a of the pulse sensor 10 emits light of the green wavelength, and this light of the green wavelength is applied to the skin of the arm through the window glass 9a of the window section 9 provided in the back cover 5, as in the case of the first embodiment. Part of this applied light of the green wavelength is absorbed by capillary vessels under the skin of the arm, and the remaining light which has not been absorbed into the capillary vessels is reflected.

This reflected light is received by the light receiving element 10b of the pulse sensor 10 through the window glass 9a of the window section 9 of the back cover 5. Then, on the basis of change in the amount of light received by this light receiving element 10b, the control section (not shown in the drawings) on the circuit board for the timepiece module calculates a pulse. The pulse information calculated thereby is sent to the circuit board for the timepiece module (not shown in the drawings) in the wristwatch case 1 through the connector 13 and the flexible wiring board (not shown in the drawings), and displayed on the display device (not shown in the drawings) of the timepiece module as a pulse rate.

As described above, the detection device 20 of the wristwatch according to the second embodiment includes the back cover 5 which is a main body case where at least part of the main surface has an opening, the sensor board 7 provided in the back cover 5, the pulse sensor 10 which is a first detection section that is provided under the sensor board 7 in the back cover 5 and detects biological information, the window section 9 which is provided under the pulse sensor 10 and is in the opening of the back cover 5, and the body wearing sensor 21 which is a second detection section that is provided on the window section 9 and detects, through the window section 9, whether or not the detection device 20 is in contact with or close to the skin of the user. As a result of this structure, the detection sensitivity of the body wearing sensor 21 serving as the second detection section can be ensured, as in the case of the first embodiment.

That is, in the case of the detection device 20 of this wristwatch, the body wearing sensor 21 is arranged without being exposed to the outside of the back cover 5, as in the case of the first embodiment. As a result, the waterproof structure is not complicated, and waterproofing is ensured by this simple waterproof structure. In addition, by being provided on the window section 9 of the back cover 5, the body wearing sensor 21 can be arranged in a non-contact state with respect to the back cover 5.

Accordingly, in the case of the detection device 20 of this wristwatch, although the back cover 5 is made of metal, an electrostatic capacity change higher than a predetermined threshold value, which occurs by the wristwatch case 1 coming in contact with or approaching a body, is unfailingly detected by the body wearing sensor 21 provided on the window section 9 of the back cover 5. As a result of this structure, whether or not the back cover 5 is in contact with a body can be unfailingly and favorably detected, whereby pulse measurement by the pulse sensor 10 is performed only when the back cover is in contact with the body, and unnecessary power consumption is prevented, as in the case of the first embodiment. In addition, since the distance between the body wearing sensor 21 and the body is shorter as compared to the first embodiment, electrostatic capacity changes can be more accurately detected.

In the case of the detection device 20 of this wristwatch, since the window section 9 of the back cover 5 has the structure where the window glass 9a has been fitted into the through hole in the back cover 5 together with the waterproof packing 9b, waterproofness is ensured with the simple waterproof structure, as in the case of the first embodiment. More specifically, this window section 9 has the structure where the through hole 15 of the back cover 5 includes the large-diameter hole 15a on the lower side and the small-diameter hole 15b on the upper side, and the window glass 9a has been fitted into the large-diameter hole 15a on the lower side via the waterproof packing 9b. Accordingly, the waterproofness of the window section 9 is ensured by the waterproof packing 9b.

Also, in the detection device 20 of this wristwatch, the body wearing sensor 21 is provided corresponding to the window glass 9a of the window section 9 of the back cover 5 while surrounding the circumference of the pulse sensor 10. As a result of this structure, the pulse sensor 10 is positioned corresponding to the portion of the window glass 9a corresponding to the small-diameter hole 15b of the through hole 15 in the window section 9 of the back cover 5, and favorably measures a pulse. In addition, although the back cover 5 is made of metal, body pulse measurement by the pulse sensor 10 is not affected thereby, and the body wearing sensor 21 favorably detects an electrostatic capacity change higher than a predetermined threshold value which occurs by the wristwatch case 1 coming in contact with or approaching a body.

Moreover, in the detection device 20 of this wristwatch, the insulative spacing member 8 is arranged between the back cover 5 which is a component mounting member and the sensor board 7. In addition, the spacing member 8 is provided with the sensor insertion hole 8a into which the pulse sensor 10 and the connection member 22 are inserted in a manner to be positioned corresponding to the window section 9. As a result of this structure, the pulse sensor 10 is arranged corresponding to the window section 9 of the back cover 5, and the body wearing sensor 21 and the connection member 22 are reliably and favorably connected to each other without the body wearing sensor 21 coming in contact with the back cover 5.

Accordingly, in the detection device 20 of this wristwatch, since the pulse sensor 10 is reliably and favorably opposed to the portion of the window glass 9a corresponding to the small-diameter hole 15b of the through hole 15 in the window section 9 of the back cover 5 by the component insertion hole 8b of the spacing member 8, a pulse is accurately measured by the pulse sensor 10, and an electrostatic capacity change higher than a predetermined threshold value, which occurs by the wristwatch case 1 coming in contact with or approaching a body, is unfailingly and favorably detected by the body wearing sensor 11.

Also, in the detection device 20 of this wristwatch, the connection member 22 to be electrically connected to the body wearing sensor 21 is provided on one surface of the sensor board 7, that is, the undersurface of the sensor board 7. As a result of this structure, although the body wearing sensor 21 is provided on the window section 9 of the back cover 5, the connection member 22 comes in contact with the body wearing sensor 21 when the sensor board 7 is arranged on the inner surface (upper surface) of the attachment recess section 5c of the back cover 5, and the body wearing sensor 21 is electrically connected to the sensor board 7 by this connection member 22.

Moreover, in the detection device 20 of this wristwatch, the connection member 22 is electrically connected to the connector 13 on the upper surface of the sensor board 7 by the wiring (not shown in the drawings) formed by pattern formation on the sensor board 7. As a result of this structure, by the connection member 22, the body wearing sensor 21 provided on the window section 9 of the back cover 5 is electrically connected to the connector 13 on the upper surface of the sensor board 7 reliably and favorably. In the present embodiment, the plurality of chip components 12, which is provided on the upper surface of the sensor board 7 and to which the pulse sensor 10 is electrically connected, is connected to the connector 13, as in the first embodiment.

Thus, in the detection device 20 of this wristwatch, the connector 13 of the sensor board 7 to which the body wearing sensor 21 and the pulse sensor 10 have been electrically connected are electrically connected to the circuit board (not shown in the drawings) for the timepiece module in the wristwatch case 1 by the flexible wiring board (not shown in the drawings). As a result of this structure, an instruction to perform pulse measurement is provided to the pulse sensor by the body wearing sensor 21, and pulse information (pulse rate) measured by the pulse sensor 10 is favorably displayed on the display device (not shown in the drawings) of the timepiece module.

In the above-described second embodiment, the connection member 22 is a spring member such as a flat spring. However, the present disclosure is not limited thereto and the connection member 22 may be a spring member such as a coil spring.

Also, in the above-described first and second embodiments, the pulse sensor 10 which detects a pulse has been described as a biosensor for these embodiments. However, the present disclosure is not limited thereto. For example, the biosensor may be an oxygen saturation sensor which optically detects oxygen saturation in a body. That is, the biosensor may be any sensor as long as it can optically detect biological information.

Moreover, in the above-described first and second embodiments, the body wearing sensors 1 and 21 are electrostatic capacity sensors which detect electrostatic capacity changes on a body. However, the present disclosure is not limited thereto. For example, the body wearing sensors 11 and 21 may be optical sensors or ultrasonic sensors.

Furthermore, in the above-described first and second embodiments, the present disclosure has been applied in a wristwatch. However, the present disclosure is not necessarily required to be applied in a wristwatch, and may be applied in a body wearable device that is used by being worn on a body part such as an arm.

Still further, in the above-described first and second embodiments, the back cover 5 of the wristwatch case 1 has been described as the component mounting member of the present disclosure. However, the present disclosure is not limited thereto. The component mounting member of the present disclosure is not necessarily be the back cover of a wristwatch case and may be the case of a body wearable device.

Yet still further, in the above-described first and second embodiments, the present disclosure has been applied in a timepiece. However, the present disclosure is not limited thereto and may be applied in a smartwatch, a smartphone, smartglasses, and a belt-type device.

While the present invention has been described with reference to the preferred embodiments, it is intended that the invention be not limited by any of the details of the description therein but includes all the embodiments which fall within the scope of the appended claims.

What is claimed is:

1. A detection device comprising:
a main body case where at least part of a main surface has an opening;
a board which is provided in the main body case;
a first detection portion which is provided under the board in the main body case and detects biological information of a user;
a window portion which is provided under the first detection portion and is in the opening of the main body case; and
a second detection portion which is provided between the board and the window portion and detects, through the window portion, whether or not the detection device is in contact with or close to the skin of the user.

2. The detection device according to claim 1, wherein the main body case includes a lower windshield member which is provided in a lower part of the main body case and has the main surface that opposes the skin of the user, and
wherein the lower windshield member is made of metal.

3. The detection device according to claim 1, wherein the second detection portion is provided on the undersurface of the board.

4. The detection device according to claim 1, wherein the second detection portion is provided to a side of the first detection portion.

5. The detection device according to claim 4, wherein the second detection portion surrounds the circumference of the first detection portion.

6. The detection device according to claim 5, wherein the first detection portion has an upper part with which the board is in contact, and a lower part with which the window portion is in contact.

7. The detection device according to claim 2, wherein an insulative spacing member is arranged between the lower windshield member and the board, and
wherein the spacing member has an insertion hole in which the first detection portion is arranged corresponding to the window portion.

8. The detection device according to claim 2, wherein the second detection portion is provided on the upper surface of the window portion.

9. The detection device according to claim 8, wherein the second detection portion surrounds the circumference of the first detection portion in a planar view from below the main body case.

10. The detection device according to claim 8, further comprising:
a connection member which electrically connects one side of the board with the second detection portion.

11. The detection device according to claim 10, wherein an insulative spacing member is arranged between the lower windshield member and the board, and
wherein the spacing member has an insertion hole in which the first detection portion and the connection member are arranged corresponding to the window portion.

12. The detection device according to claim 1, wherein the first detection portion is a pulse sensor which has a light emitting portion and a light receiving portion, and detects a pulse.

13. The detection device according to claim 1, wherein the second detection portion is an electrostatic capacity sensor which detects an electrostatic capacity change that occurs by the detection device coming in contact with or approaching the skin of the user.

14. The detection device according to claim 13, wherein the second detection portion is formed of metal.

15. The detection device according to claim 1, wherein the window portion, the first detection portion, and the second detection portion oppose the skin of the user when the detection device is used by the user.

16. A timepiece comprising the detection device according to claim 1.
